(19) United States
(12) Patent Application Publication
(10) Pub. No.: US 2008/0262081 A1
(43) Pub. Date: Oct. 23, 2008

(54) Novel Use of Nutraceutical Compositions Comprising Resveratrol

(76) Inventors: Daniel Raederstorff, Flaxlanden (FR); Ying Wang-Schmidt, Stallikon (CH); Swen Wolfram, Waldshut-Tiengen (DE)

Correspondence Address: NIXON & VANDERHYE, PC, 901 NORTH GLEBE ROAD, 11TH FLOOR, ARLINGTON, VA, 22203, US

(21) Appl. No.: 12/089,584

(22) PCT Filed: Oct. 11, 2006
(86) PCT No.: PCT/EP2006/009815

§ 371 Date: Jun. 13, 2008

(30) Foreign Application Priority Data
Oct. 14, 2005 (EP) 05022413.8
May 15, 2006 (EP) 06009927.2

(51) Int. Cl. A61K 31/05; A61K 31/366

(52) U.S. Cl. 514/457; 514/733; 514/458

(57) ABSTRACT

The use of resveratrol, a derivative, metabolite or analogue thereof, in combination with at least one additional component selected from EGCG, coenzyme Q-IO, genistein, lycopene, hydroxytyrosol and polyunsaturated fatty acids in the manufacture of a nutraceutical composition for delaying aging and/or for the treatment or prevention of age-related diseases in animals, in particular in mammals including humans.

The present invention relates to a novel use of nutraceutical compositions comprising as active ingredients, resveratrol, a derivative, metabolite or analogue thereof, and at least one additional component selected from EGCG, coenzyme Q-10, genistein, lycopene, hydroxytyrosol and polyunsaturated fatty acids.

More specifically, the invention relates to the use of such nutraceutical compositions for delaying aging and/or for the treatment or prevention of age-related diseases in animals, in particular in mammals including humans.

The term “nutraceutical” as used herein denotes usefulness in both the nutritional and pharmaceutical field of application. Thus, the novel nutraceutical compositions can find use as supplement to food and beverages, dietary supplement and as pharmaceutical formulations for enteral or parenteral application which may be solid formulations such as capsules or tablets, or liquid formulations, such as solutions or suspensions. As will be evident from the foregoing, the term nutraceutical composition also comprises food and beverages containing the above-specified active ingredients.

The term “resveratrol, a derivative, metabolite or analogue thereof” as used herein comprises compounds encompassed by the general formula

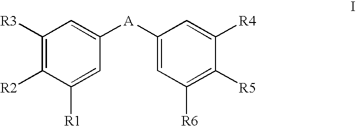

wherein A denotes a carbon-carbon single or double bond which latter may be trans or cis, and R1, R2, R3, R4, R5 and R6, independently from each other denote hydrogen, hydroxy, etherified hydroxy, esterified hydroxy groups. Preferred compounds I wherein A is a double bond (—CH═CH—).

Etherified or esterified hydroxy groups may be derived from unsubstituted or substituted, straight or branched chain alkyl groups having 1 to 26 carbon atoms or from unsubstituted or substituted, straight or branched chain aliphatic, araliphatic or aromatic carboxylic acids having 1 to 26 carbon atoms. Etherified hydroxy groups may further be glycoside groups and esterified hydroxy groups may further be glucuronide or sulfate groups. Examples of compounds of formula I wherein A is —CH═CH— are resveratrol (R1, R3 and R5=hydrogen, R2, R4 and R6=hydroxy); piceatannol (R3 and R5=hydrogen, R1, R2, R4 and R6=hydroxy), and rhapontigenin (R5=hydrogen, R1, R3, R4 and R6=hydroxy, and R2=methoxy). Examples of compounds of formula I wherein A is —CH₂—CH₂— are dihydroresveratrol (R1, R3 and R5=hydrogen; R2, R4 and R6=hydroxy), dihydropiceatannol (R3 and R5=hydrogen; R1, R2, R4 and R6=hydroxy) and tristin (R3 and R5=hydrogen; R2, R4 and R6=hydroxy and R1=methoxy). These compounds are all wellknown and commercially available or can be obtained in accordance with methods well-known in the art.

The term “EGCG” as used herein comprises (−)-epigallocatechin gallate (EGCG) and/or one or more derivatives (esterified forms, glycosides, sulphates) thereof. EGCG is the major catechin found in green tea. The beneficial health effects of green tea have been mainly attributed to the catechins. In mice, tea catechins reduced diet-induced weight gain, visceral fat mass, as well as plasma leptin, triglyceride, and glucose levels. Tea catechins are also known to increase energy expenditure in rats. In humans, tea catechins have been shown to reduce body weight, visceral fat mass, and plasma cholesterol, insulin, and glucose levels. Green tea extract was shown to significantly increase energy expenditure and fat oxidation in healthy men. Furthermore, it was shown in brown adipose tissue of rats that EGCG stimulates metabolic activity and oxygen consumption. Additionally, several animal studies demonstrated that catechins inhibited cholesterol absorption and lowered plasma cholesterol levels. In turn, epicatechins increase the fecal excretion of cholesterol and total lipids.

Coenzyme Q-10, (6-Decaprenyl-2,3-dimethoxy-5-methyl-1,4-benzoquinone) is a fat soluble quinone with a structure similar to vitamin K. The health beneficial effects of Coenzyme Q10 (CoQ10) have been associated with its two main biochemical functions. CoQa10 is an essential cofactor of the mitochondrial electron transport chain which, is coupled to synthesis of adenosine triphosphate (ATP). Therefore, it acts as a catalyst in the biochemical pathway that leads to cellular energy production. This bioenergic effect of CoQ10 is of particular importance in cells with high metabolic demands such as cardiac myocytes. Moreover, CoQ10 is an important antioxidant in both the mitochondria and lipid membranes. CoQ10 exerts a sparing effect on vitamin E and has membrane stabilizing properties. Several studies showed that LDL oxidation was reduced after CoQ10 supplementation. Thus, CoQ10 may improve energy metabolism and protect against oxidative stress in diabetes and cardiovascular diseases.

The term “genistein” as used herein comprises the aglycone (4′,5,7-trihydroxyisoflavone) and derivatives thereof, e.g., genistein glycosides, genistein sulfates, genistein glucuronides. Genistein is a phytoestrogen belonging to the isoflavone class of flavonoid. It is abundant in soy bean and was reported to have antioxidant activities.

Lycopene (ψ,ψ carotene; C₄₀H₅₆; CAS-number: 502-65-8) belongs to the carotenoid family containing 11 conjugated double-bonds and in addition two non-conjugated carbon-carbon double-bonds. Lycopene is one of the major dietary carotenoids and is found in various fruits and vegetables, especially in tomatoes and tomato products. It also occurs, e.g., in water melon, pink grapefruit, guava.

The term “hydroxytyrosol” as used herein comprises hydroxytyrosol (3,4-dihydroxyphenylethanol) and/or one or more derivatives (esterified forms, glycosides, sulphates) or a molecule containing hydroxytyrosol such as for example oleuropein an heteroside ester of elenolic acid and hydroxytyrosol or oleuropein aglycones or verbascoside ((caffeic acid-glucose-(rhamnose)-hydroxytyrosol). Hydroxytyrosol or one of its derivatives or analogues is in the form of a purified plant extract especially an olive extract. Hydroxytyrosol is the main polyphenol found in olives. Hydroxytyrosol is believed to be the antioxidant with the highest free radical scavenging capacity: double that of quercetin and more than 3 times that of epicatechin. The wastewaters generated during olive processing contain a high levels of hydroxytyrosol, most of which can be recovered to produce hydroxytyrosol extracts. Hydroxytyrosol has the same health promoting properties than other polyphenols: prevention of atherosclerosis, promotion of intestinal and respiratory health and prevention of cancer. Hydroxytyrosol also reduces the oxidative stress caused by smoking.

The term “polyunsaturated fatty acids” as used herein (herein also referred to as PUFA) denotes a polyunsaturated fatty acid in an esterified (e.g., as triglycerides or ethyl esters) or a free form, particularly an omega-3 polyunsaturated fatty acid such as eicosapentaenoic acid (5,8,11,14,17-eicosapentaenoic acid, EPA) and docosahexaenoic acid (4,7,10,13,16,19-docosahexaenoic acid, DHA), or an omega-6-polyunsaturated fatty acid such as y-linolenic acid (6,9,12-octadecatrienoic, GLA).

Aging involves a progressive deterioration and loss of the cellular processes and physiological functions of an organism that ultimately increase the likelihood of death. The proportion of the aging population is increasing worldwide. Therefore, there is an urgent need for developing interventions that delay aging and the age-related diseases, which retard the deterioration of certain body functions and improve the quality of life and life expectancy of older peoples. The aging process involves a number of molecular pathways such as oxidative stress, cellular stress resistance, neuroendocrine systems, nutrient sensing systems and insulin signaling. Recent research suggests that the nutrient sensing systems and the insulin signaling pathways play a key role in the aging process. Moreover, the modulation of the insulin signaling pathways and the nutrients sensing systems can delay age related diseases. Finally both the nutrients sensing systems and the insulin signaling pathways seems to be conserved through evolution and across species as diverse as yeast and humans. The insulin signaling pathways have been shown to play a critical role in the regulation of lifespan and also of growth and size in different species. Moreover, insulin sensitivity is linked with the development of certain age-related diseases such as diabetes and cancer. Thus, modulation of the insulin pathway can lengthen lifespan of drosophila and mice having effects on longevity across species. Finally, recent studies showed that centenarians have generally an efficient insulin response. The nutrient sensing systems are also conserved from the unicellular yeast to mammals and have been linked to the aging process and to age-related diseases such as diabetes and cancer.

There are several nutrient sensing systems that detect energy and metabolic status and adjust nutrient flux by promoting an anabolic phenotype in times of plenty and/or a catabolic phenotype in time of starving. The mammalian target of rapamycin-S6 kinase (mTOR-S6K) pathway is one of the nutrient sensing systems. The protein S6 kinase (S6K1) is involved in the control of protein translation and the regulation of cell growth and proliferation. Repression of S6 kinase extended the lifespan of drosophila. In the contrary, overexpression of S6 kinase decreased the drosophila lifespan. Recently, knocking out S6K1 in mouse was shown to protect mice from age- or diet-induced obesity and enhance insulin sensitivity. Moreover, mice on a high fat diet and obese ob/ob mice have markedly elevated S6K1 activity. S6K1 activation has been suggested to mediate some of the effects of a high-fat diet and to make people become less sensitive to insulin as they age. S6K1 mediates insulin signaling and also plays a role in nutrient sensing pathways. It seems that repression of S6K1 may protect the organisms from the deleterious effects of overeating. Thus, targeting S6K1 may be a way to counteract aging and age-related diseases such as neurodegenerative diseases (Alzheimer, dementia), atherosclerosis, cardiovascular diseases, cancer, diabetes and obesity.

In the 1950s, it was very unlikely that one would live to be more than 100 years old, or even 90. At that time, 35 was considered middle age. However, less than half a century later, the age group of 85 and above is the fastest growing population in the United States. While people are living longer, there are also more diseases and other disorders in the aging population. Thus, there is urgent need for health- and wellness-promoting measures in order to delay the aging process and to reduce the incidence of age-related diseases. Healthy diet and lifestyle changes greatly improve the health status and the quality of life of the aging population. Nutraceutical compositions with beneficial effects on age-related pathological changes may help to restrain aging and solve the medical problems caused by the rapidly increasing aging population.

Diseases and other disorders in the aging population can be grouped as follows:

Central nervous system disorders: The aging process often causes atrophic changes in the brain. There are substantial age-related declines in brain function, i.e., decrease in norepinephrine and dopamine synthesis. Some neurons gradually die in the brain; however, others will grow to compensate for the age-related deaths of their neighbors, similar to what happens in hippocampus. There are also age-related neurological and psychiatric disorders such as Alzheimer, depression.

Autonomic nervous system disorders: Since the homeostatic mechanisms slow and weaken during advancing age, changes are reflected in the alterations of sympathetic and parasympathetic responsiveness, i.e., decreased sensitivity of baroreceptor and change in thermoregulation. Consequently, orthostatic hypotension and syncope are common problems for the elderly and are only worsened by disease, especially diabetic autonomic dysfunction.

Eye and ear disorders: Eye Disorders - Physiological changes of presbyopia and lens opacification subsequently cause decreased accommodation and increased susceptibility to glare. These physiological changes often result in decreased visual acuity as well as blindness. Ear Disorders - For the ear, the physiological change is decreased high frequency acuity, making it difficult to discriminate words if noise is present in the background. Consequently, there is deafness and a decrease in acoustic acuity.

Other groups of diseases and disorders in the aging population are:
Cardiovascular system disorders (diseases include hypertension, coronary artery disease, congestive heart failure as well as heart block or arrhythmia);
Respiratory system disorders (Respiratory diseases include emphysema, dyspnea, and hypoxia);
Gastrointestinal system disorders (the elderly may have hepatic cirrhosis, constipation, fecal impaction, fecal incontinence, osteoporosis or vitamin B12 deficiency due to poor absorption);
Endocrine system disorders (include the development of diabetes mellitus, thyroid dysfunction);
Hematological and immune system disorders (the development of anemia and autoimmune disease);
Muscular and skeletal system disorders (osteoporosis);
Cancer;

Surprisingly, it has been found that compositions containing the active ingredients, resveratrol, a derivative, metabolite or analogue thereof, and at least one additional component selected from EGCG, coenzyme Q-10, genistein, lycopene, hydroxytyrosol or polyunsaturated fatty acids may be useful for delaying the aging process and/or for the treatment or prevention of age-related diseases in animals, in particular in mammals including humans.

Groups of animals of particular interest apart from mammals and humans in connection with the present invention are, e.g. domestic animals and pets, such as horses, camels, dromedaries, dogs, cats and birds, and animals kept in zoological gardens.

Domestic animals, pets and zoo animals will receive the active ingredients preferably via their food, e.g., via pet food, including their drinking water.

Moreover, it has been found that the present compositions act on different critical signaling pathways involved in aging and hence delay aging and age-related diseases more potently than the individual components. Thus, the present composition can delay the process of aging in part by acting on nutrient sensing and insulin signaling pathways which are linked to aging and longevity. Especially, the present compositions regulate the mTOR-S6K1 pathway and hence delay the aging of a cell or an organism by altering the nutrient sensing systems and the insulin signaling pathways.

The nutraceutical compositions of the present invention contain resveratrol, a derivative, metabolite or analogue thereof in an amount sufficient to provide to a human adult (weighing about 70 kg) a dosage from about 0.5 mg/day to about 2000 mg/day, preferably from about 5 mg/day to about 500 mg/day. Thus, if the nutraceutical composition is a food or beverage the amount of a resveratrol compound contained therein is suitably in the range from about 0.2 mg to about 500 mg per serving. If the nutraceutical composition is a pharmaceutical formulation such formulation may contain from about 0.5 mg to about 500 mg per solid dosage unit, e.g., per capsule or tablet, or from about 0.5 mg per daily dose to about 2000 mg per daily dose of a liquid formulation.

EGCG is preferably used in a concentration so that the daily consumption by a human adult (weighing about 70 kg) is in the range of from 10 mg/day to 2000 mg/day. A food or beverage suitably contains about 2 mg to about 500 mg of EGCG per serving. If the nutraceutical composition is a pharmaceutical formulation such formulation may contain a EGCG in an amount from about 5 mg to about 500 mg per dosage unit, e.g., per capsule or tablet, or from about 10 mg per daily dose to about 2000 mg per daily dose of a liquid formulation.

The amount of hydroxytyrosol in the composition may be such to provide a daily dosage from about 0.01 mg per kg body weight to about 60 mg per kg body weight of the subject to which it is to be administered. A food or beverage suitably contains about 0.3 mg per serving to about 1250 mg per serving of hydroxytyrosol. If the nutraceutical composition is a pharmaceutical formulation such formulation may contain hydroxytyrosol in an amount from about 1 mg to about 4000 mg per dosage unit, e.g., per capsule or tablet, or from about 1 mg per daily dose to about 4000 mg per daily dose of a liquid formulation.

PUFA's are preferably used in a concentration so that the daily consumption by a human adult (weighing about 70 kg) is in the range of from 10 mg/day to 4000 mg/day. A food or beverage suitably contains about 5 mg to about 1000 mg of a PUFA per serving. If the nutraceutical composition is a pharmaceutical formulation such formulation may contain a PUFA in an amount from about 10 mg to about 1000 mg per dosage unit, e.g., per capsule or tablet, or from about 10 mg per daily dose to about 4000 mg per daily dose of a liquid formulation.

Genistein is preferably used in a concentration so that the daily consumption by a human adult (weighing about 70 kg) is in the range of from 0.5 mg/day to 2000 mg/day. A food or beverage suitably contains about 0.2 mg to about 500 mg of genistein per serving. If the nutraceutical composition is a pharmaceutical formulation such formulation may contain a genistein in an amount from about 0.5 mg to about 500 mg per dosage unit, e.g., per capsule or tablet, or from about 0.5 mg per daily dose to about 2000 mg per daily dose of a liquid formulation.

Lycopene is preferably used in a concentration so that the daily consumption by an animal including humans (e.g. weighing about 70 kg) is in the range of from 0.05 mg/day to 50 mg/day (corresponding to a daily dosage of about 0.0007 to about 0.7 mg/kg body weight), more preferably from 0.5 mg/day to 30 mg/day. A nutraceutical composition preferably comprises 0.05 mg to 50 mg of lycopene per serving. If the composition is a pharmaceutical composition such composition may preferably comprise lycopene in an amount from 0.5 mg to 50 mg per dosage unit, e.g., per capsule or tablet, or a liquid formulation unit.

The term “serving” as used herein denotes an amount of food or beverage normally ingested by a human adult with a meal at a time and may range, e.g., from about 100 g to about 500 g.

The active ingredients of the composition defined above have different mechanisms of action thus providing synergistic effects in preventing age-related diseases.

In a preferred embodiment of the invention the composition comprises a combination of EGCG and resveratrol or a combination of hydroxytyrosol and resveratrol. Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

In one aspect of the present invention the compositions may be used as nutritional supplements, e.g., as additives to multi-vitamin preparations comprising vitamins and minerals which are essential for the maintenance of normal metabolic functions but which are not synthesized in the body, especially for the treatment or prevention of age-related diseases.

Specific combinations of active ingredients in the compositions of the present invention comprise

Resveratrol and EGCG;

Resveratrol and Hydroxtyrosol;

Resveratrol and at least one PUFA (such as EPA, DHA, GLA);

Resveratrol and genistein;

Resveratrol and lycopene;

Resveratrol and CoQ10.

The following Examples illustrate the invention further.

### A. Pharmaceutical Compositions may be Prepared by Conventional Formulation Procedures Using the Ingredients Specified Below:

### EXAMPLE 1

Soft gelatin capsule

Soft gelatin capsules are prepared by conventional procedures using ingredients specified below:

Active ingredients: Resveratrol 10 mg, EPA 200 mg, vitamin E 50 mg

Other ingredients: glycerol, water, gelatine, vegetable oil.

### EXAMPLE 2

Hard gelatin capsule

Hard gelatin capsules are prepared by conventional procedures using ingredients specified below:

Active ingredients: resveratrol 10 mg, EGCG 100 mg, genistein, 5 mg, vitamin E 50 mg, vitamin K 1 mg

Other ingredients: Fillers: lactose or cellulose or cellulose derivatives, Lubricant: magnesium stearate if necessary (0.5%)

### EXAMPLE 3

Tablet

Tablets are prepared by conventional procedures using ingredients specified below:

Active ingredients: resveratrol 5 mg, EGCG 50 mg, vitamin E 20 mg

Other ingredients: microcrystalline cellulose, silicone dioxide (SiO2), magnesium stearate, crosscarmellose sodium.

B. Food Items may be Prepared by Conventional Procedures using Ingredients Specified Below:

### EXAMPLE 4

Soft Drink with 30% juice

Active ingredients: Resveratrol and one or more additional components selected from EGCG, PUFA (EPA; DHA; GLA), genistein, vitamin E and vitamin K are incorporated in this food item:

Resveratrol: 0.2-200 mg/ per serving

EGCG: 2-200 mg/ per serving

PUFA (EPA; DHA, GLA): 5-500 mg/ per serving

Genistein: 0.2-50 mg/ per serving

Vitamin E: 5-100 mg/ per serving

Vitamin K: 0.01-5 mg/ per serving

Typical serving: 240 ml

I. A Soft Drink Compound is Prepared from the following Ingredients:

| | [g] |
|---|---|
| 1.1. Orange concentrate | |
| 60.3 °Brix, 5.15% acidity | 657.99 |
| Lemon concentrate | |
| 43.5 °Brix, 32.7% acidity | 95.96 |
| Orange flavour, water soluble | 13.43 |
| Apricot flavour, water soluble | 6.71 |
| Water | 26.46 |
| 1.2 Color | |
| β-Carotene 10% CWS | 0.89 |
| Water | 67.65 |
| 1.3 Acid and Antioxidant | |
| Ascorbic acid | 4.11 |
| Citric acid anhydrous | 0.69 |
| Water | 43.18 |
| 1.4 Stabilizers | |
| Pectin | 0.20 |
| Sodium benzoate | 2.74 |
| Water | 65.60 |
| 1.5 Oil soluble flavours | |
| Orange flavour, oil soluble | 0.34 |
| Orange oil distilled | 0.34 |

1.6 Active Ingredients

Active ingredients (this means the active ingredients mentioned above: resveratrol and one or more of the following EGCG, PUFA (EPA; DHA; GLA), genistein, vitamin E and vitamin K) in the concentrations mentioned above.

Fruit juice concentrates and water soluble flavours are mixed without incorporation of air. The color is dissolved in deionized water. Ascorbic acid and citric acid is dissolved in water. Sodium benozoate is dissolved in water. The pectin is added under stirring and dissolved while boiling. The solution is cooled down. Orange oil and oil soluble flavours are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenising using a Turrax and then a high-pressure homogenizer (p₁=200 bar, p₂=50 bar).

II. A Bottling Syrup is Prepared from the following Ingredients:

| | [g] |
|---|---|
| Soft drink compound | 74.50 |
| Water | 50.00 |
| Sugar syrup 60° Brix | 150.00 |

The ingredients of the bottling syrup are mixed together. The bottling syrup is diluted with water to 11 of ready to drink beverage.

Variations:

Instead of using sodium benzoate, the beverage may be pasteurised. The beverage may also be carbonised.

### EXAMPLE 5

Cookies Type Milano

Active ingredients: Resveratrol and one or more additional components selected from EGCG, PUFA (EPA; DHA; GLA), genistein, vitamin E and vitamin K are incorporated in this food item:

Resveratrol: 0.2-100 mg/ per serving

EGCG: 2-100 mg/ per serving

PUFA (EPA; DHA, GLA): 5-200 mg/ per serving

Genistein: 0.2-20 mg/ per serving

Vitamin E: 5-100 mg/ per serving

Vitamin K: 0.01-5 mg/ per serving

Typical serving: 30 g

| | [g] |
|---|---|
| Wheat Flour, type 550 | 41.0 |
| Sugar | 20.5 |
| Fat/Butter | 20.5 |
| Whole egg (liquid) | 18.0 |
| Lemon Flavour | q.s. |
| Baking agent | q.s. |

All ingredients are added slowly under mixing to form a sweet short pastry.

Afterwards, the pastry is kept cool (4° C.) for at least 2 hours before flattening the pastry to a thickness of approx. 5 mm. Pieces are cut out and brushed with egg yolk on the surface before baking.

Baking:

| | |
|---|---|
| Oven: | fan oven |
| Baking temperature: | 180° C. |
| Baking time: | 15 min |

C. The Effect of EGCG and Resveratrol in C3H101/2 cells:

Mouse embryonic mesenchymal stem cells, C3H10T1/2 cells, (ATCC, USA, #CCL-226), were cultured in DMEM (low glucose 1 g/ml, Gibco), supplemented with 10% FBS and 2 mM L-Glutamine (Gibco). 2×10⁵ cells /well were seeded in 6-well plate and grown to 80% confluency. Cells were treated with either rapamycin (Tocris, UK, #1292) at 100 nM, EGCG (TEAVIGO®, DSM Nutritional Product ltd. Switzerland) at 10 microM , resveratrol (DSM Nutritional Products Ltd. Switzerland) at 0.1, 1, 10 μM or combination of EGCG (10 microM) and resveratrol (0.1,1, 10 μM) for 30 min, then stimulated with insulin (Sigma, Switzerland) at 100 nM for 30 min. Cells were washed twice with PBS and harvested in 0.4 ml NETT buffer (0.1 M NaCl, 10 mM Tris-HCL (pH 7.6), 1 mM EDTA, 1% Triton X-100, pH 7.6 ). Cell lysates were sonicated and centrifuged at 14,000 rpm for 2 min. Protein concentration was determined using BCA assay (PIERCE, USA #23223). 10 microgram whole cell extracts were loaded on SDS-gel (10-20% Tricine gradient gel) and blotted on nitrocellulose membrane (0.2 μm pore, Invitrogen, #LC2000 ). Blot was incubated first with monoclonal antibody, phosphor-p70 S6 Kinase (Thr389)(1A5) mouse mAb (Cell signaling technology, #9206) at 1:2000 dilution, then with secondary antibody, goat anti-mouse IgG HRP (Santa Cruz, USA, #SC2055) at 1:10000. Protein load was control by measuring β-actin using anti mouse β-actin (Sigma, Switzerland, #A-5441). The signals were detected using ECL plus Western Blotting Detection System (Amersham Biosciences; RPN2132). Membrane was exposed to X-ray film for 30′ (Hyperfilm ECL; High performance chemiluminescence film, Amersham Biosciences; RPN1674 ) and films were scanned with ChemiGenius 2 machine (Syngene, UK). The signals were quantified by using GeneTools software (Syngene, UK). Signal of phosphorylated p70S6K was normalized to the protein load control β-actin, and vehicle was set up as 100%.

Results

The mammalian target of rapamycin (mTOR) is part of nutrient sensing pathway and controls multiple cellular functions in response to amino acids and growth factors. It is activated by amino acid starvation, as well as growth factors, such as insulin, IGF-1 and IGF-II, and regulates cell-cycle progression and cell growth. In presence of nutrient and mitogens, mTOR stimulates protein synthesis and cell growth via its downstream target, including the 4E-BP12 (eukaryotic initiation factor 4E-binding protein) and the S6K kinase. p70S6K is a protein serine-threonine kinase that phosphorylates ribosomal S6 subunit, and therefore plays a key role in protein synthesis and cell growth. Insulin modulates p70S6K through its downstream target Akt, which in turn activates mTOR and p7OS6k. p70S6K regulates also cell proliferation. It was postulated that mTOR-S6K plays key role in sensing energy status and maintain balance between fuel usage of amino acid and glucose. In mice, increased mTOR activity is associated with development of cancer, diabetes and obesity. Signal of mTOR pathways are significantly elevated in liver and skeletal muscle of insulin-resistance obese rats. Knockout of p7OS6k, mTOR's downstream target protects mice from diet-induced and obesity and improve insulin sensitivity. Reduced mTOR-S6K signaling also extends life span of yeast, drosophila, and C. elegans.

TABLE I
Phosphorylated p70S6K (Thr389) level from treated C3H10T1/2 cells.

| | % vs. | |
|---|---|---|
| Samples | Vehicle | % CV |
| Vehicle (DMSO) | 100 | 12.305 |
| Rapa 100 nM | * | * |
| 10 μM EGCG; | 117.20 | 17.167 |
| 0.1 μM Resveratrol | 95.23443 | 27.88562 |
| 1 μM Resveratrol | 106.9157 | 3.570731 |
| 10 μM Resveratrol | 132.715 | 16.1571 |
| 10 μM EGCG& 0.1 μM Resveratrol | 76.14655 | 23.845 |
| 10 μM EGCG & 1 μM Resveratrol | 55.52649 | 1.247 |
| 10 μM EGCG & 10 μM Resveratrol | 57.16629 | 0.006 |

* under detectable level

The effects of natural polyphenol, EGCG and resveratrol, on mTOR-p70S6K pathway were studied in C3H101/2 cells. Rapamycin, a known mTOR inhibitor, was used as positive control. We observed that 100 nM rapamycin treatment totally abolished phophorylated p70S6K (Thr389). EGCG treatment at low dose, 10 microM, had little effect on amount of phosphorylated p70S6K. Resveratrol treatment, at 0.1, 1 and 10 microM also has no influence on phosphorylated p70S6K amount, if at all, showed a dose-dependent increase of p70S6K(Thr389). Surprisingly, combination of EGCG (10 microM) and resveratrol (0.1,1, 10 microM) more than synergistically reduced phosphorylated p70S6K amount. Reduced mTOR-p70S6K signalling has been associated with expanded lifespan in yeast, drosophila and C. elegans, as well as reduced risk for diet-induced obesity and enhanced insulin sensitivity in mice. The synergistic effect of EGCG and resveratrol on inhibition of phosphorylated p70S6K, and thus, reducing signalling of mTOR-S6K pathway, indicates that these two natural polyphenols may beneficial influence ageing process and prevent age-related pathological changes.

1. The use of resveratrol, a derivative, metabolite or analogue thereof, in combination with at least one additional component selected from EGCO, coenzyme Q-10, genistein, lycopene, hydroxytyrosol and polyunsaturated fatty acids in the manufacture of a nutraceutical composition for delaying aging and/or for the treatment or prevention of age-related diseases in animals, in particular in mammals including humans.

2. The use of resveratrol, a derivative, metabolite or analogue thereof, in combination with at least one additional component selected from EGCG, coenzyme Q-10, genistein, hydroxytyrosol and polyunsaturated fatty acids in the manufacture of a nutraceutical composition for delaying aging and/or for the treatment or prevention of age-related diseases in mammals, in particular in humans.

3. The use as in claim 1, wherein said resveratrol is used in an amount sufficient to provide a daily dosage of 0.03 mg per kg body weight to about 10 mg per kg body weight of the subject to which it is to be administered; said EGCG is used in an amount sufficient to provide a daily dosage of 0.1 mg per kg body weight to about 10 mg per kg body weight of the subject to which it is to be administered; said lycopene is used in an amount sufficient to provide a daily dosage of 0.0007 mg per kg body weight to about 0.7 mg per kg body weight of the subject to which it is to be administered; said polyunsaturated fatty acid is used in an amount sufficient to provide a daily dosage of 1.0 mg per kg body weight to about 50 mg per kg body weight of the subject to which it is to be administered, said genistein is used in an amount sufficient to provide a daily dosage of 0.03 mg per kg body weight to about 10 mg per kg body weight of the subject to which it is to be administered.

4. The use as in claim 1, wherein the nutraceutical composition is a food or beverage, or a supplement composition for food or beverage.

5. The use as in claim 1, wherein the nutraceutical composition is a pharmaceutical composition.
